(19) United States
(12) Patent Application Publication
(10) Pub. No.: US 2023/0135826 A1
(43) Pub. Date: May 4, 2023

(54) Difluoromethylene Synthons And Methods Of Use

(71) Applicant: The Trustees of the University of Pennsylvania, Philadelphia, PA (US)

(72) Inventors: Amos B Smith, III, Merion Station, PA (US); Kevin T O'Brien, Sicklerville, NJ (US); Jonathan W Nadraws, Philadelphia, PA (US)

(21) Appl. No.: 18/051,549

(22) Filed: Nov. 1, 2022

Related U.S. Application Data
(60) Provisional application No. 63/274,591, filed on Nov. 2, 2021.

(51) Int. Cl. C07F 7/08; C07D 307/20; C07F 7/10

(52) U.S. Cl. CPC C07F 7/081; C07D 307/20; C07F 7/10

(57) ABSTRACT

Organodifluorine synthons, in conjunction with three-component diastereoselective Anion Relay Chemistry (ARC), permit ready access to diverse difluoromethylene scaffolds. Initiated via [1,2]-addition of an organolithium reagent to a β-difluoromethylene silyl aldehyde, an alkoxide intermediate is formed, which is capable of undergoing a [1,4]-Brook rearrangement to generate a stabilized α-difluoromethylene carbanion, which upon electrophile capture, affords a three-component adduct. This three-component synthetic tactic represents an important one-pot divergent strategy for the construction of diverse organodifluorine containing compounds.

### CROSS-REFERENCE TO RELATED APPLICATIONS

The present application claims the benefit of priority to U.S. Provisional Application No. 63/274,591, filed Nov. 2, 2021, the entire contents of which are incorporated herein by reference.

### GOVERNMENT RIGHTS

This invention was made with government support under Contract No. CHE-1827457 awarded by the National Science Foundation, and under Contract No. CA-19033 awarded by the National Institutes of Health. The government has certain rights in the invention.

### TECHNICAL FIELD

The present disclosure relates to compounds for use in preparing difluoromethylene scaffolds.

### BACKGROUND

Organofluorine compounds are ubiquitous in modern society, with polytetrafluoroethylene (PTFE) polymers and hydrofluorocarbon (HFC) refrigerants seeing widespread use.¹ Over the last several decades, the role of fluorine in small molecule therapeutics has gained significant attention, with approximately 20% of approved active pharmaceutical ingredients and 35% agrochemicals containing at least one fluorine atom.² The strong polarization of the C—F bond, in conjunction with the short bond length and small Van der Waals radius of the fluorine atom, results in a variety of useful properties.³ In medicinal chemistry, these properties manifest in myriad ways, permitting fluorine to serve as a bioisostere,⁴ to modify the basicity and acidity of adjacent functional groups,⁵ to effect the solubility of compounds,⁶ to alter metabolism,⁷ and/or to control the conformation of structures,⁸ among other attributes.

Accordingly, numerous methods for the incorporation of fluorine atoms into organic compounds have been developed, which utilize either direct or indirect fluorination tactics, either adding a single fluorine atom, for instance employing DAST or SelectFluor, or an activated organofluorine reagent such as the Ruppert-Prakash reagent (TMS-CF₃).⁹ However, most existing fluorination methods employ mono-valent synthons, which in general permit only linear syntheses. Ideally, fluorination could be performed as a convergent synthetic step, permitting the reaction to serve as a point of diversification, which is often desirable from the perspective of the medicinal chemist. Recently, Dilman has exploited di-valent difluoromethylene synthons for use in multi-component coupling tactics (FIG. 1b).¹⁰ While highly effective, this method lacks a broad scope of coupling partners and requires multiple steps to achieve.

### SUMMARY

Disclosed herein are organodifluorine compounds according to Formula (I)

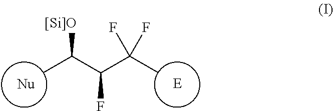

wherein Nu represents a nucleophile, E represents an electrophile, and R is a linchpin substrate.

Also provided are methods for preparing an organodifluorine synthon of Formula (I).

The present disclosure also provides difluoromethylene scaffolds that are prepared using the disclosed organodifluorine synthons of Formula (I), as well as organodifluorine-containing compounds that are prepared using such scaffolds.

### BRIEF DESCRIPTION OF THE DRAWINGS

FIG. 1A depicts fluorination synthons, FIG. 1B illustrates a prior coupling tactic employing a dianion synthon, and FIG. 1C represents a scheme according to the present disclosure, involving a fluoorganic synthon and a three-component coupling tactic via Type II Anion Relay Chemistry (ARC).

FIG. 2 represents a stepwise evaluation of an Anion Relay Chemistry (ARC) sequence that was used to develop the present synthons.

FIG. 3 depicts results of an investigation into the exemplary scope of the electrophile (E) group in the present synthons.

FIG. 4 depicts results of an investigation into the exemplary scope of the nucleophile (Nu) group in the present synthons.

FIG. 5 depicts results of an investigation into the exemplary scope of the linchpin substrate (R) in the present synthons.

### DETAILED DESCRIPTION OF ILLUSTRATIVE EMBODIMENTS

The present invention may be understood more readily by reference to the following detailed description taken in connection with the accompanying examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

The disclosures of each patent, patent application, and publication cited or described in this document are hereby incorporated herein by reference, in their entirety.

As employed above and throughout the disclosure, the following terms and abbreviations, unless otherwise indicated, shall be understood to have the following meanings.

In the present disclosure the singular forms “a”,“an”, and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. Furthermore, when indicating that a certain chemical moiety “may be” X, Y, or Z, it is not intended by such usage to exclude other choices for the moiety; for example, a statement to the effect that R₁ “may be alkyl, aryl, or amino” does not exclude other choices for R₁, such as halo, aralkyl, and the like.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” may refer to a value of 7.2 to 8.8, inclusive; as another example, the phrase “about 8%” may refer to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” In another example, when a listing of possible substituents including “hydrogen, alkyl, and aryl” is provided, the recited listing may be construed as including situations whereby any of “hydrogen, alkyl, and aryl” is negatively excluded; thus, a recitation of “hydrogen, alkyl, and aryl” may be construed as “hydrogen and aryl, but not alkyl”, or simply “wherein the substituent is not alkyl”.

Protective groups are abbreviated according to the system disclosed in Greene, T. W. and Wuts, P. G. M, Protective Groups in Organic Synthesis 2d. Ed., Wiley & Sons, 1991, which is incorporated in its entirety herein. For example, “CBZ” or “Cbz” or “Z” stands for carbobenzyloxy or benzyloxycarbonyl, “Boc” or “BOC” represents t-butoxycarbonyl, “Alloc” denotes allyloxycarbonyl, Bz means benzoyl, and “Fmoc” stands for 9-fluorenylmethoxycarbonyl.

As used herein, the terms “component,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, “alkyl” refers to an optionally substituted, saturated straight, or branched, hydrocarbon radical having from about 1 to about 20 carbon atoms (and all combinations and subcombinations of ranges and specific numbers of carbon atoms therein). Where appropriate, “alkyl” can mean “alkylene”; for example, if X is —R₁R₂, and R₁ is said to be “alkyl”, then “alkyl” may correctly be interpreted to mean “alkylene”.

“Amino” refers to —NH₂ and may include one or more substituents that replace hydrogen. “Amino” may be used interchangeably with “amine” and is also intended to include any pharmaceutically acceptable amine salts. For example, amino/amine may refer to —NH⁺(X)(Y)Cl⁻, —NH—, or —NH⁺(X)Cl⁻—, wherein X and Y are preferably and independently hydrogen or alkyl, wherein alkyl may include one or more halo substitutions.

As used herein, “aryl”, “arene”, and “aromatic” each refer to an optionally substituted, saturated or unsaturated, monocyclic, polycyclic, or other homo- or heterocyclic aromatic ring system having from about 3 to about 50 ring members (and all combinations and subcombinations of ranges and specific numbers of carbon atoms therein), with from about 5 to about 10 ring atom members being preferred. Such moieties encompass (include) “heteroaryl” and “heteroarene” as defined infra. Where appropriate, “aryl” can mean “arene”; for example, if X is —R₁R₂, and R₁ is said to be “aryl”, then “aryl” may correctly be interpreted to mean “arene”.

As used herein, “alkenyl” refers to an alkyl radical having from about 2 to about 20 carbon atoms and one or more double bonds (and all combinations and subcombinations of ranges and specific numbers of carbon atoms therein), wherein alkyl is as previously defined. In some embodiments, it is preferred that the alkenyl groups have from about 2 to about 6 carbon atoms. Alkenyl groups may be optionally substituted.

As used herein, “aralkyl” refers to alkyl radicals bearing one or more aryl substituents and having from about 4 to about 50 carbon atoms (and all combinations and subcombinations of ranges and specific numbers of carbon atoms therein), wherein aryl and alkyl are as previously defined. In some preferred embodiments, the alkyl moieties of the aralkyl groups have from about 1 to about 4 carbon atoms. In other preferred embodiments, the alkyl moieties have from about 1 to about 3 carbon atoms. Aralkyl groups may be optionally substituted.

“Alkylamino” signifies alkyl-(NH)—, wherein alkyl is as previously described and NH is defined in accordance with the provided definition of amino. “Arylamino” represents aryl-(NH)—, wherein aryl is as defined herein and NH is defined in accordance with the provided definition of amino. Likewise, “aralkylamino” is used to denote aralkyl-(NH)—, wherein aralkyl is as previously defined and NH is defined in accordance with the provided definition of amino. “Alkylamido” refers to alkyl-CH(═O)NH—, wherein alkyl is as previously described. “Alkoxy” as used herein refers to the group R—O— where R is an alkyl group, and alkyl is as previously described. “Aralkoxy” stands for R—O—, wherein R is an aralkyl group as previously defined. “Alkylsulfonyl” means alkyl-SO₂—, wherein alkyl is as previously defined.

The present have developed organodifluorine synthons, in conjunction with three-component diastereoselective Anion Relay Chemistry (ARC), that permit ready access to diverse difluoromethylene scaffolds. In the synthetic scheme, initiated via [1,2]-addition of an organolithium reagent to a β-difluoromethylene silyl aldehyde, an alkoxide intermediate is formed, which is capable of undergoing a [1,4]-Brook rearrangement to generate a stabilized α-difluoromethylene carbanion, which upon electrophile capture, affords a three-component adduct. This three-component synthetic tactic represents a novel one-pot divergent strategy for the construction of diverse organodifluorine containing compounds.

Accordingly, provided herein are organodifluorine synthons of Formula (I):

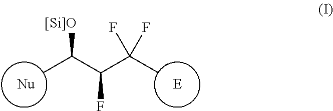

wherein Nu represents a nucleophile, E represents an electrophile, and R is a linchpin substrate. In some embodiments, R is alkyl, alkenyl, or aryl. In certain embodiments, Nu is aryl, alkenyl, aryl, aralkyl, or aralkenyl. E may be, for example, aryl, alkenyl, aryl, aralkyl, aralkenyl, or alkylsulfoxyaminoalkyl. Other specific examples of R, Nu, and E are described in the present disclosure, but are not intended to be limiting to the possible choices for these groups. In fact, those of ordinary skill in the art may select any nucleophilic group for Nu, any electrophilic group for E, and any desired group for R, according to the desired use of the organodifluorine synthon.

Also provided herein are methods for preparing an organodifluorine synthon of Formula (I). The method may comprise performing [1,2]-addition of an organolithium reagent (Nuc) to a β-difluoromethylene silyl aldehyde in order to form an alkoxide intermediate; and, subjecting the alkoxide intermediate to [1,4]-Brook rearrangement to generate a stabilized α-difluoromethylene carbanion; wherein when the α-difluoromethylene carbanion undergoes electrophile (E) capture the organodifluorine synthon of Formula (I) is formed. In some embodiments, the method for preparing an organodifluorine synthon of Formula (I) is according to Scheme I:

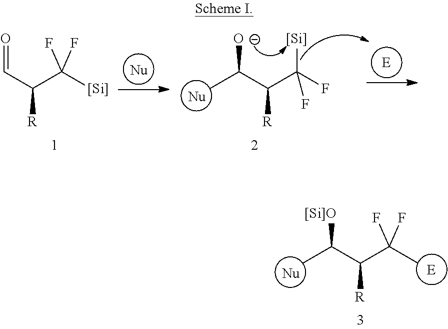

Also disclosed are difluoromethylene scaffolds that are prepared using an organodifluorine synthon according to Formula (I). The present difluoromethylene scaffolds may be prepared using one organodifluorine synthon according to Formula (I), or may be prepared using two or more different organodifluorine synthons according to Formula (I).

The present disclosure also provides organodifluorine-containing compounds that are prepared using the present difluoromethylene scaffolds.

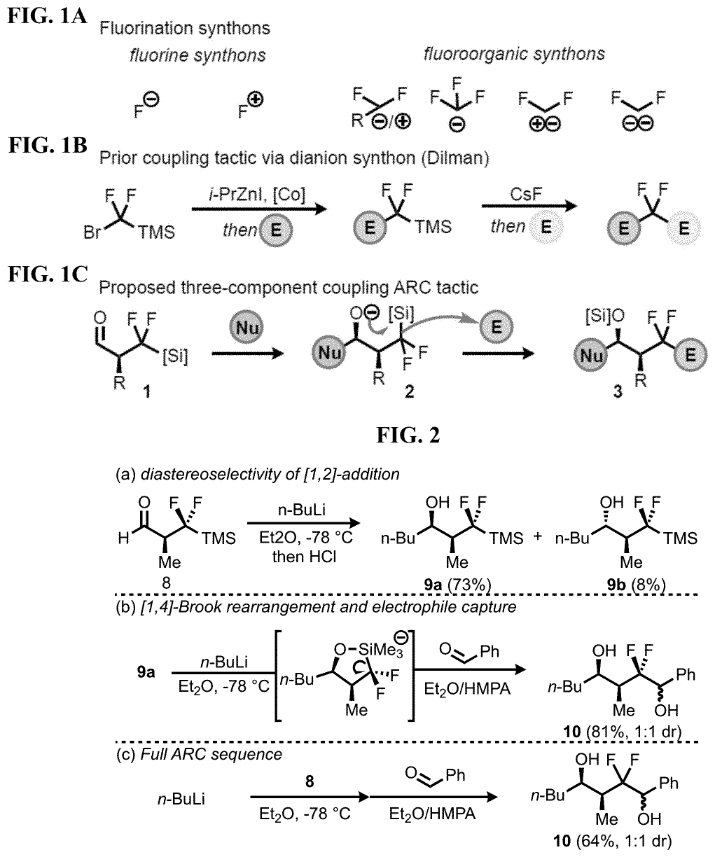

As provided above, prior fluorination methods have employed mono-valent synthons, which in general permit only linear syntheses (FIG. 1A), or by exploiting di-valent difluoromethylene synthons for use in multi-component coupling tactics (FIG. 1B). The present inventors have developed a one-pot three-component union (FIG. 1C) that employs an ambiphilic linchpin in which the anionic carbon is stabilized by geminal fluorine substituents, in a tactic comprising [1,2]-addition of an organolithium reagent to aldehyde linchpin 1 to afford an alkoxide intermediate (2). Subsequent [1,4]-Brook rearrangement of alkoxide 2 with electrophile capture then affords a three-component adduct (3), with the relative stereochemistry of the Anion Relay Chemistry (ARC) adduct under Felkin-Anh stereocontrol.¹¹ Benefits associated with the present synthetic approach include that (a) the scope of each component is broad, (b) the diastereoselectivity is high, (c) the reaction is performed in a single pot, and (d) the linchpin is readily constructed.

### EXAMPLES

The present invention is further defined in the following Examples. It should be understood that these examples, while indicating preferred embodiments of the invention, are given by way of illustration only, and should not be construed as limiting the appended claims From the above discussion and these examples, one skilled in the art can ascertain the essential characteristics of this invention, and without departing from the spirit and scope thereof, can make various changes and modifications of the invention to adapt it to various usages and conditions.

### Example 1—Development of Organodifluorine Synthons

Work was conducted in order to validate a synthetic route to access diverse difluoro-linchpins (1), which would be amenable to varied substitution at the α-position. The present inventors sought to construct β-difluoro-linchpins via a direct α-silyldifluoromethylation of an oxazolidinone derived lithium enolate. Initially, oxazolidinone 4 was acylated with an acid chloride to afford 5 (Scheme 1). Next, the lithium enolate of 5 was generated with LiHMDS and subsequently α-silyldifluoro-methylated, to furnish 6 as a single diastereomer. The absolute stereochemistry of 6 was determined unambiguously by X-ray crystallography (Supporting Informtion). The desired linchpin was then obtained via reduction of oxazolidinone 6 with LiAlH4 to provide 7, followed by oxidation with Dess-Martin Periodinane (DMP) to afford 8 in good yield.

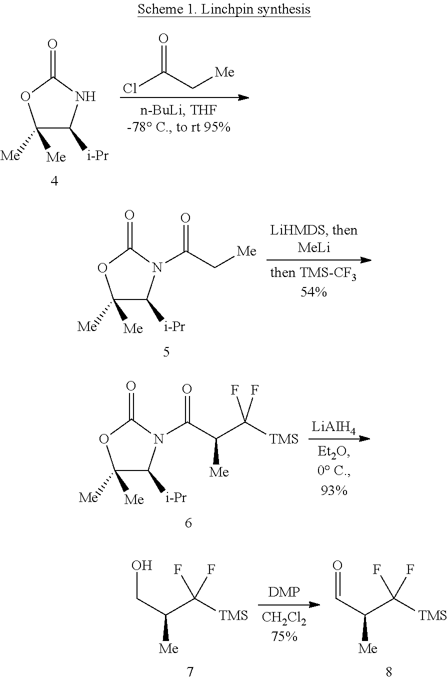

With the desired linchpin 8 in hand, the next objective was to validate the proposed reaction sequence (FIG. 2). High diastereoselectivity of the initial [1,2]-addition was deemed critical for the utility of this transformation. The inventors thus first performed the initial [1,2]-addition of n-BuLi to linchpin 8 at −78° C. in Et₂O, with subsequent reaction with HCl in Et₂O to arrive at alcohols 9a and 9b as separable diastereomers. High diastereoselectivity was observed (9:1 by ¹H-NMR analysis), with the absolute configuration of 9a determined by Mosher ester analysis, demonstrating that Felkin-Anh diastereoselectivity was operative.¹³ Next, deprotonation of 9a was achieved with n-BuLi at −78° C. in Et₂O, and benzaldehyde added. The envisioned [1,4]-Brook rearrangement was then triggered via the use of the polar additive HMPA. Pleasingly, tricomponent adduct 10 was obtained in 72% yield as an expected 1:1 mixture of diastereomers at the benzylic position. Having demonstrated the proof-of-concept via a two-pot ARC sequence utilizing 9a, we next turned our attention to validation of the one-pot, three-component coupling ARC tactic. To this end, employing the defined conditions, three-component adduct 10 was observed as the major product from the one-pot sequence in 64% yield.

A further general synthetic procedure using a phenyl oxaxolidinone auxiliary was carried out as shown below in accordance with Scheme S-2:

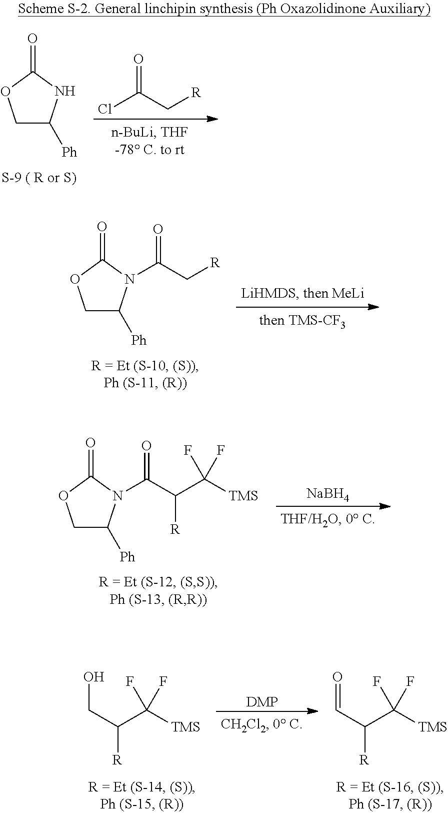

### Example 2—Electrophile (E) Scope

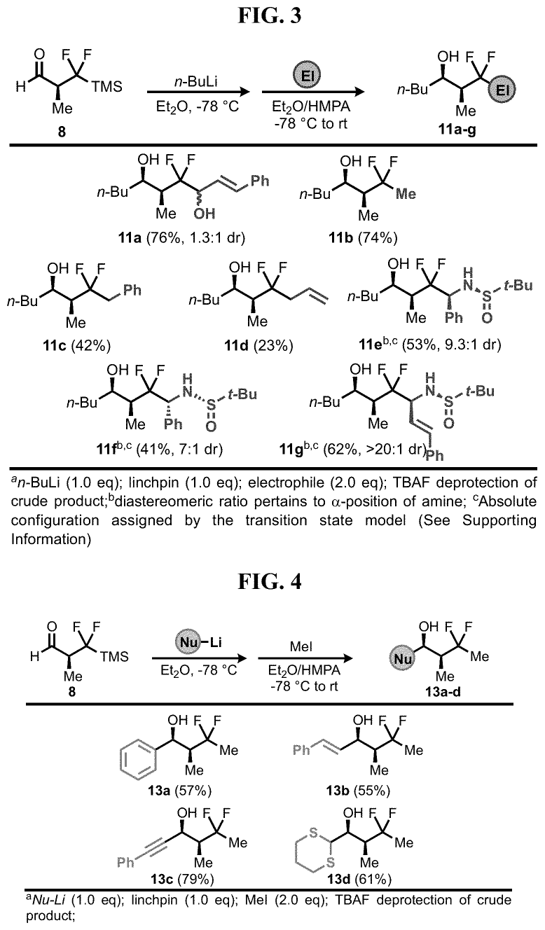

Having validated the ARC sequence, the next objective was to explore the electrophile scope. Reactions were performed using n-BuLi as the nucleophile and linchpin 8, with a variety of electrophiles (FIG. 3). Evaluation of (E)-cinnamaldehyde as an electrophile demonstrated that α,β-unsaturated aldehyde electrophiles are amendable to the ARC sequence to provide allyl alcohol 11a. Next, a series of alkyl halide electrophiles were employed, with MeI affording 11b in good yield and benzyl and allyl bromide affording three-component adducts 11e and 11d in modest yield. Pleasingly, enantioenriched sulfonyl imine electrophiles also served as successful electrophiles in the ARC sequence, providing β-difluoroamine substrates as three-component adducts as single diastereomers; both (S)- and (R)-phenyl sulfonyl amine furnished ARC adducts 11e and 11f with high diastereoselectivity in moderate yield. Additionally, employing the (R)-α,β-unsaturated sulfonyl imine of cinnamaldehyde permitted access to β-difluoro-allyl amine 11g in 62% yield. Thus, this ARC tactic holds the promise of a viable method for the construction of enantioenriched β-difluoroamines, for which limited methods are currently available.¹⁴

At first glance it may appear that the three-component adducts obtained from aldehyde electrophiles are less than ideal, as there is no diastereoselectivity in the second [1,2]-addition. However, such diol products can be used to access difluorocyclopentyl ketals (Scheme 2). Similar pseudo-pentose structural motifs are found in a number of bioactive compounds, such as the approved drug Gemcitabine (Gemzar™). For example, benzylic oxidation of 10 with MnO₂ leads spontaneously to ketal formation to arrive at 12, as a 4:1 mixture of diastereomers in excellent yield. A similar strategy may be adapted to permit the synthesis of other difluorocyclopentyl ketals from the corresponding aldehyde adducts.

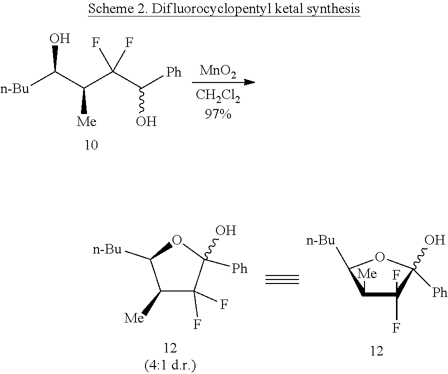

### Example 3—Nucleophile Groups

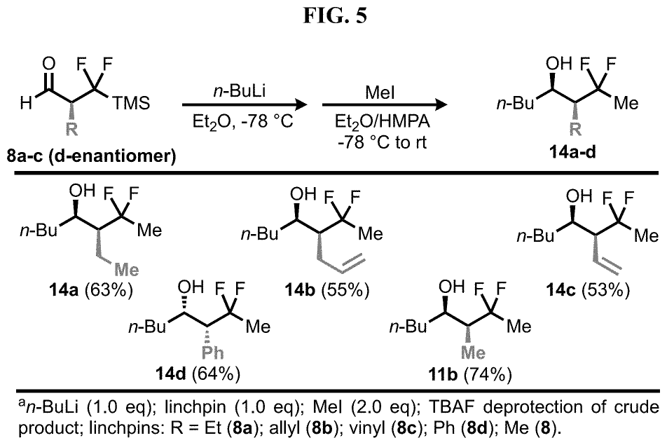

The next objective was to investigate the scope of organolithium reagents that could be employed as initiating nucleophiles in this ARC sequence. Standard reaction conditions were employed, utilizing linchpin 8 to evaluate a variety of organolithium reagents (FIG. 4). Having demonstrated that n-butyllithium with methyl iodide as the electrophile is amenable to the ARC tactic, commercially available PhLi was devleoped. Accordingly, the three-component adduct 13a was obtained as a single diastereomer in moderate yield. Vinyl- and alkynyllithium reagents were also successfully employed to afford allyl- and propargyl alcohols 13b and 13c in moderate to good yield. Equally pleasingly, lithiated 1,3-dithiane can be utilized successfully in the ARC sequence, permitting the incorporation of this versatile functionality in adduct 13d. Thus, alkyl, vinyl, alkynyl, aryl, and dithianyllithium reagents comprise viable nucleophiles in this three-component ARC coupling tactic.

### Example 4—Linchpin Substrates

Another significant aspect of this coupling tactic would be the tolerance on variability of the linchpin. Initially, the inventors had examined exclusively the viability of linchpin 8, bearing an α-Me substitution. While this substitution is useful for polyketide synthetic targets, larger substituents or functional handles would serve to provide access to a more diverse pool of difluoromethylene scaffolds. The next objective was therefore to examine several linchpins with varied α-substitution. For the evaluation of linchpins 8a-8d, standard reaction conditions were employed, utilizing n-BuLi as the nucleophile and MeI as the electrophile. Increasing the size of the substituent from Me to Et had a small negative effect on yield, arriving at the three-component adduct 14a in 63% yield. Olefin moieties were well tolerated at the α-position, affording homoallyl and allyl products 14b and 14c. Pleasingly, Ph and vinyl α-substitutions were also well tolerated, despite the expected increased acidity of the corresponding linchpins. Furthermore, the synthesis of three-component adduct 14d demonstrates that this ARC methodology is amenable to both epimers of the linchpins.

A number of synthetic methods are predicated upon the manipulation of difluoromethyl radicals.¹⁵ The σ-withdrawing, π-donating properties of the fluorine atom permit access to difluoromethyl radicals, anions,¹⁶ and carbenes.¹⁷ Mechanistic studies were performed to provide support in this case for an anionic reaction mechanism for the [1,4]-Brook rearrangement with alkylation (Scheme 3). Here, the principle concern was the [1,4]-Brook rearrangement, rather than the [1,2]-carbonyl addition. Thus, the ARC sequence was entered via the deprotonation of alcohol 9a. Next, the [1,4]-Brook rearrangement was triggered via the addition of a solution of benzaldehyde in Et₂O/HMPA, with or without an equivalent of TEMPO. In both cases, three-component adduct 10 was obtained in good yield. Additionally, TEMPO adduct 15 was not identified in the trapping experiment. Next, to offer support for a [1,4]-Brook rearrangement we demonstrated the isolation of silyl ether ARC product 16. The labile nature of TMS ethers led us to remove this group prior to purification for simplicity. However, isolation of 16 here demonstrates that C—Si to O—Si migration indeed occurs.

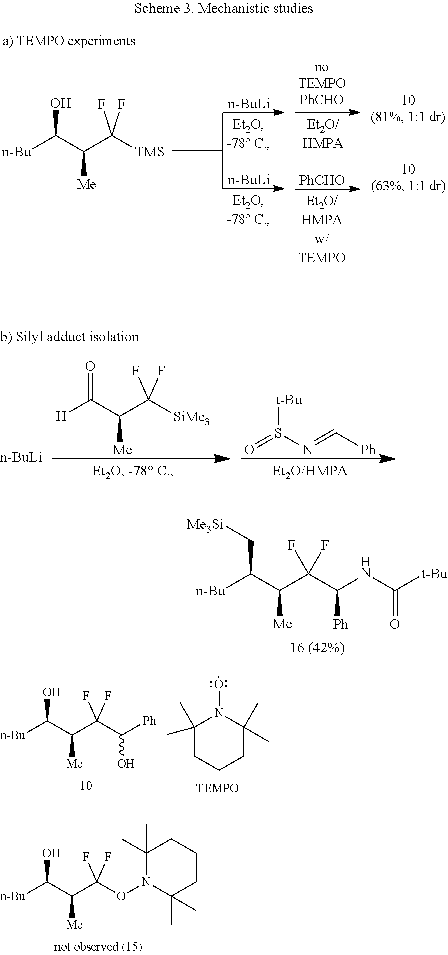

In summary, the present inventors have developed new ambiphilic organodifluoromethylene synthon which can be employed in a three-component ARC coupling tactic to afford a variety of difluoromethylene adducts with high diastereoselectivity. Moreover, we have disclosed a synthetic route that permits access to these β-difluorosilyl aldehyde linchpins in which the α-substituent and absolute stereochemical configuration can be readily achieved by selecting the appropriate acid chloride and oxazolidione substrates, respectively, many of which are commercially available. The value of this synthon and the corresponding three-component coupling tactic is also apparent in the great variety of nucleophiles, linchpins, and electrophiles that can be employed. Thus, it is possible to prepare numerous difluoromethylene scaffolds employing this tactic as a key disconnection, which may otherwise be laborious to construct.

Additional information concerning the present invention is appended hereto as Appendix I.

### REFERENCES

Superscripted numbers in the preceding text refer to the correspondingly numbered references as follows:

(1) (a) Puts, G. J., Crouse, P., Ameduri, B. M. Polytetrafluoroethylene: Synthesis and Characterization of the Original Extreme Polymer. Chem. Rev. 2019, 119(3), 1763-1805; (b) Hurwitz, M. M., Fleming, E. L., Newman, P. A., Li, F., Mlawer, E., Cady-Pereira, K., Bailey, R. Ozone depletion by hydrofluorocarbons. Geophys. Res. Lett. 2015, 42 (20), 8686-8692.

(2) O'Hagan, D. Understanding Organofluorine Chemistry: An Introduction to the C—F Bond. Chem. Soc. Rev. 2008, 37, 308-319.

(3) Wang, J. Sanchez-Rosello, M., Acena, J. L., del Pozo, C., Sorochinsky, A. E., Fustero, S., Soloshonok, V. A., Liu, H. Fluorine in Pharmaceutical Industry: Fluorine-Containing Drugs Introduced to the Market in the Last Decade (2001-2011). Chem. Rev. 2014, 114(4), 2432-2506.

(4) Edmondson, S. D., Wei, L., Xu, J. Shang, J., Xu, S., Pang, J., Chaudhary, A., Dean, D. C., He, H., Leiting, B., Lyons, K. A., Patel, R. A., Patel, S. B., Scapin, G., Wu, J. K., Beconi, M. G., Thornberry, N. A., Weber, A. E. Fluoroolefines as amide bond mimics in dipeptidyl peptidase IV inhibitors. Bioorg. and Med Chem. Lett. 2008, 18(7), 2409-24213.

(5) Qiu, J., Stevenson, S. H., O'Beirn, M. J., Silverman, R. B. 2,6-Difluorophenol as a Bioisostere of a Carboxylic Acid: Bioisosteric Analogues of γ-Aminobutyric Acid. J. Med. Chem. 1999, 42(2), 329-332.

(6) Ettorre, A., D'Andrea, P., Mauro, S., Porcelloni, M., Rossi, C., Altamura, M., Catalioto, R. M., Giuliani, S., Maggi, C. A., Fattori, D. hNK₂ receptor antagonists. The use of intramolecular hydrogen bonding to increase solubility and membrane permeability. Bioorg. and Med Chem. Lett. 2011, 21(6), 1807-1809.

(7) Park, B. K., Kitteringham, N. R., O'Neil, P. M. Metabolism of Fluorine-Containing Drugs. Ann. Rev. of Pharmacol. and Toxic. 2001, 41, 443-470.

(8) Wu, D., Tian, A., Sun, H. Conformational Properties of 1,3-Difluoropropane. J. Phys. Chem. A. 1998, 102(48), 9901-9905.

(9) (a) Ruppert, I., Schlich, K., Volbach, W. The first CF₃-substituted chloroorganosilane. Tet. Lett. 1984, 25(21), 2195-2198. (b) Prakash, G. K. S., Yidin, A. K. Perfluoroalkylation with Organosilicon Reagents. Chem. Rev. 1997, 97(3), 757-786.

(10) Kosobokov, M. D., Levin, V. V., Zemstov, A. A., Struchkova, M. I., Korlyukov, A. A., Arkhipov, D. E., Dilman, A. D. Geminal Silicon/Zinc Reagent as an Equivalent of Difluoromethylene Bis-carbanion. Org. Lett. 2014, 16(5), 1438-1441.

(11) (a) Cherest, M.; Felkin, H.; Prudent, N. Torsional Strain Involving Partial Bonds. The Stereochemistry of Lithium Aluminum Hydride Reduction of Simple Open-Chain Ketones. Tet. Lett. 1968, 9(18), 2199-2204. (b) Anh, N.; Lefour, J M.; Dau, T. Torsional Strain Involving Partial Bonds. The Stereochemistry of Lithium Aluminum Hydride Reduction of Simple Open-Chain Ketones. Tet. Lett. 1968, 9(18), 2199-2204.

(12) Hashimoto, R.; Iida, T.; Aikawa, K, K.; Ito, S.; Mikami, K. Direct α-Siladifluoromethylation of Lithium Enolates with Ruppert-Prakash Reagent via C—F Bond Activation. Chem. Eur. J. 2014, 20(10), 2750-2754.

(13) Hoye, T. R., Jeffrey, C. S., Shao, F. Mosher ester analysis for the determination of absolute configuration of stereogenic (chiral) carbinol carbons. Nat. Protoc. 2007, 2451-2458.

(14) Fadeyi, O. O. Lindsley, C. W. Rapid, General Access to Chiral β-Fluoroamines and β,β-Difluoroamines via Organocatalysis. Org. Lett. 2009, 11(4), 943-946.

(15) Heine, N. B., Studer, A. Radical Difluoromethylation of Thiols with (Difluoromethyl)triphenylphosphonium Bromide. Org. Lett. 2017, 19(15), 4150-4153.

(16) Prakash, G. K. S., Hu, J., Wang, Y., Olah, G. A. Nucleophilic Difluoromethylation of Primary Alkyl Halides Using Difluoromethyl Phenyl Sulfone as a Difluoromethyl Anion Equivalent. Org. Lett. 2004, 6(23), 4315-4317.

(17) Brahms, D. L. S., Dailey, W. P. Fluorinated Carbenes. Chem. Rev. 1996, 96(5), 1585-1632.

What is claimed:

1. An organodifluorine synthon of Formula (I): 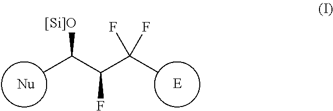
wherein Nu represents a nucleophile, E represents an electrophile, and R is a linchpin substrate.

2. The synthon according to claim 1, wherein R is alkyl, alkenyl, or aryl.

3. The synthon according to claim 1, wherein Nu is aryl, alkenyl, aryl, aralkyl, or aralkenyl.

4. The synthon according to claim 1, wherein E is aryl, alkenyl, aryl, aralkyl, aralkenyl, or alkylsulfoxyaminoalkyl.

5. A method for preparing an organodifluorine synthon of Formula (I): 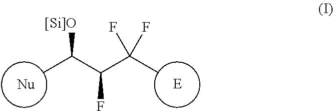
comprising:
performing [1,2]-addition of an organolithium reagent (Nuc) to a β-difluoromethylene silyl aldehyde in order to form an alkoxide intermediate;
subjecting the alkoxide intermediate to [1,4]-Brook rearrangement to generate a stabilized α-difluoromethylene carbanion;
wherein when the α-difluoromethylene carbanion undergoes electrophile (E) capture the organodifluorine synthon of Formula (I) is formed.

6. The method according to claim 5, wherein the organodifluorine synthon is prepared

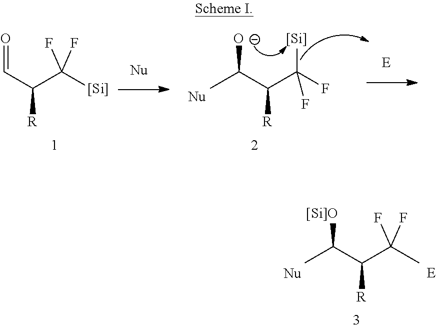

7. A difluoromethylene scaffold that is prepared using the organodifluorine synthon according to any one of claims 1.

8. An organodifluorine-containing compound that is prepared using the scaffold according to claim 7.
